(19) United States
(12) Patent Application Publication
(10) Pub. No.: US 2022/0265247 A1
(43) Pub. Date: Aug. 25, 2022

(54) ULTRASONIC IMAGE DIAGNOSTIC DEVICE, DISPLAY CONTROL METHOD, AND DISPLAY CONTROL PROGRAM

(71) Applicant: Konica Minolta, Inc., Tokyo (JP)

(72) Inventor: Takashi KIMOTO, Yokohama-shi (JP)

(21) Appl. No.: 17/649,871

(22) Filed: Feb. 3, 2022

(30) Foreign Application Priority Data
Feb. 25, 2021 (JP) 2021-028726

(51) Int. Cl. A61B 8/00

(52) U.S. Cl. CPC A61B 8/54; A61B 8/468; A61B 8/463

(57) ABSTRACT

An ultrasonic image diagnostic device includes: a transmitter/receiver that drives an ultrasonic probe to transmit an ultrasonic wave into a subject and receives the ultrasonic wave from an inside of the subject to output reception data; an ultrasonic image generator that generates an ultrasonic image on the basis of the output reception data; an imaged image generator that inputs imaged data output from an imager that images a periphery of the subject, and generates an imaged image; and a hardware processor that simultaneously displays the generated ultrasonic image and imaged image in real time.

The entire disclosure of Japanese patent Application No. 2021-028726, filed on Feb. 25, 2021, is incorporated herein by reference in its entirety.

### BACKGROUND

### Technological Field

The present invention relates to an ultrasonic image diagnostic device, a display control method, and a display control program.

### Description of the Related Art

An ultrasonic image diagnostic device that transmits and receives ultrasonic waves to and from a subject such as a living body by an ultrasonic probe (probe), generates ultrasonic image data on the basis of a signal obtained from the received ultrasonic wave, and displays an ultrasonic image based on the same on an image display device is conventionally known. Ultrasonic image diagnosis by the ultrasonic image diagnostic device is such that a state such as a heartbeat or movement of a fetus may be obtained in real time by simple operation of just putting the ultrasonic probe on a body surface of the subject, and this is noninvasive and safe, so that this may be repeatedly performed.

The diagnosis and recording of the ultrasonic image by the ultrasonic image diagnostic device are performed as follows. That is, the ultrasonic probe is put on the body surface of the subject, processing such as analog-digital conversion and image reconfiguration is performed on a reflected wave obtained by scanning the inside of the subject with an ultrasonic beam, and an ultrasonic image is displayed on a monitor (display). A user (operator) stores a desired image by a predetermined operation while observing the ultrasonic image displayed on the monitor.

The ultrasonic image is stored with a schematic view of each site of the subject (hereinafter, a body mark). The body mark includes a probe mark for enabling an observer to grasp a direction and a site of abutment of the ultrasonic probe to the subject when obtaining the image in a case where the ultrasonic image is observed later.

In a normal ultrasonic image diagnostic device, the body marks of a plurality of patterns are registered in advance. The operator may select and use an appropriate body mark from a plurality of registered body marks. In a case where the appropriate body mark is not registered, a new body mark may be created.

JP 2001-112752 A discloses a technology capable of easily using an appearance image in which appearance information such as a subject, a site of the subject, and a position of a probe is accurately imaged as information regarding an ultrasonic image. In the technology disclosed in JP 2001-112752 A, in a case where a desired ultrasonic image is captured during observation of the ultrasonic image by real-time display, when the operator performs a freeze operation to fix ultrasonic image display, the freeze operation and imaging of the appearance image are interlocked. The appearance image imaged in conjunction with the freeze operation of the ultrasonic image is combined with the frozen ultrasonic image and displayed on the monitor.

As described above, in the technology disclosed in JP 2001-112752 A, the appearance image imaged in conjunction with the freeze operation of the ultrasonic image is combined with the frozen ultrasonic image and displayed on the monitor. However, from the viewpoint of improving the accuracy of the ultrasonic diagnosis, in a case where the operator wants to grasp an operation status of the probe and a status of the subject, that is, information inside and outside the subject in real time in addition to the displayed ultrasonic image, it is necessary to directly visually confirm the operation status of the probe and the status of the subject, and there is a problem that it takes time and effort.

### SUMMARY

An object of the present invention is to provide an ultrasonic image diagnostic device, a display control method, and a display control program capable of easily grasping information inside and outside the subject in real time.

To achieve the abovementioned object, according to an aspect of the present invention, an ultrasound diagnostic device reflecting one aspect of the present invention comprises a transmitter/receiver that drives an ultrasonic probe to transmit an ultrasonic wave into a subject and receives the ultrasonic wave from an inside of the subject to output reception data; an ultrasonic image generator that generates an ultrasonic image on the basis of the output reception data; an imaged image generator that inputs imaged data output from an imager that images a periphery of the subject, and generates an imaged image; and a controller that simultaneously displays the generated ultrasonic image and imaged image in real time.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an appearance view of an ultrasonic image diagnostic device;

FIG. 2 is a block diagram illustrating a functional configuration of the ultrasonic image diagnostic device;

and

FIG. 3 is a view illustrating an execution timing of each operation in the ultrasonic image diagnostic device.

### DETAILED DESCRIPTION OF EMBODIMENTS

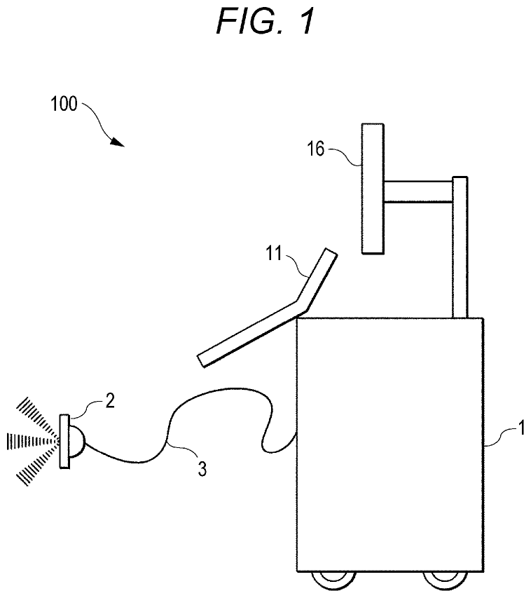

Hereinafter, one or more embodiments of the present invention will be described with reference to the drawings. However, the scope of the invention is not limited to the disclosed embodiments. FIG. 1 is an appearance view of an ultrasonic image diagnostic device 100.

As illustrated in FIG. 1, the ultrasonic image diagnostic device 100 is provided with an ultrasonic image diagnostic device main body 1, an ultrasonic probe 2, an imager 4, and a voice inputter 5. Note that the ultrasonic image diagnostic device main body 1 serves as an “ultrasonic image diagnostic device” of the present invention.

The ultrasonic probe 2 transmits an ultrasonic wave (transmission ultrasonic wave) to an inside of a subject such as a living body not illustrated, and receives a reflected wave of the ultrasonic wave (reflected ultrasonic wave: echo) reflected in the subject.

The ultrasonic image diagnostic device main body 1 is connected to the ultrasonic probe 2 via a cable 3, and transmits a drive signal of an electric signal to the ultrasonic probe 2 to cause the ultrasonic probe 2 to transmit the transmission ultrasonic wave to the subject.

The ultrasonic image diagnostic device main body 1 images an inner state in the subject as an ultrasonic image on the basis of a reception signal that is an electric signal generated by the ultrasonic probe 2 according to the reflected ultrasonic wave from the inside of the subject received by the ultrasonic probe 2. The ultrasonic image diagnostic device main body 1 is provided with an operation inputter 11 and a display 16 to be described later.

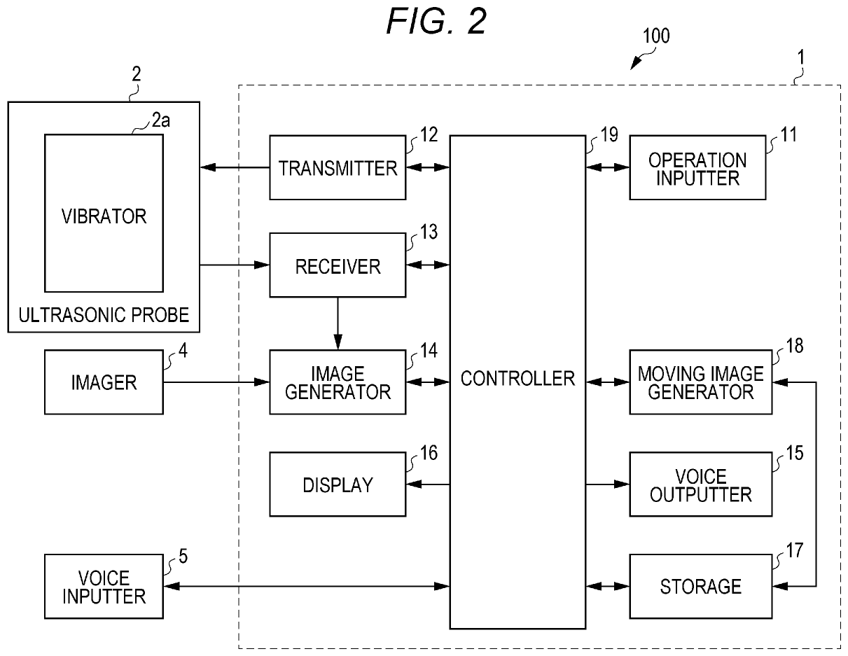

The ultrasonic probe 2 is provided with a vibrator 2a (refer to FIG. 2) formed of a piezoelectric element. A plurality of vibrators 2a is arranged in a one-dimensional array in an azimuth direction (scanning direction), for example. In this embodiment, for example, the ultrasonic probe 2 provided with 192 vibrators 2a is used.

Note that the vibrators 2a may be arranged in a two-dimensional array. The number of vibrators 2a may be arbitrarily set. In this embodiment, a linear electronic scan probe is used as the ultrasonic probe 2 to perform ultrasonic scanning by a linear scanning system, but any system of a sector scanning system or a convex scanning system may be adopted. Communication between the ultrasonic image diagnostic device main body 1 and the ultrasonic probe 2 may be performed by wireless communication such as ultra wide band (UWB) instead of wired communication via the cable 3.

The imager 4 formed of, for example, a CCD camera, images a periphery of the subject. Then, each time the imager 4 images the periphery of the subject, this outputs imaged data indicating the imaged periphery of the subject to the ultrasonic image diagnostic device main body 1.

The voice inputter 5 formed of, for example, a microphone collects voice around the ultrasonic image diagnostic device 100, and receives an input of the voice. Then, each time the voice inputter 5 receives the input of the voice, this outputs voice data indicating the voice the input of which is received to the ultrasonic image diagnostic device main body 1 (controller 19). The controller 19 acquires the voice data output from the voice inputter 5 and causes a storage 17 to be described later to store the same. Note that the controller 19 serves as a “voice data acquirer” of the present invention.

Next, a functional configuration of the ultrasonic image diagnostic device 100 is described with reference to FIG. 2. FIG. 2 is a block diagram illustrating the functional configuration of the ultrasonic image diagnostic device 100.

As illustrated in FIG. 2, the ultrasonic image diagnostic device main body 1 is provided with, for example, the operation inputter 11, a transmitter 12, a receiver 13, an image generator 14, a voice outputter 15, the display 16, the storage 17, a moving image generator 18, and the controller 19.

The operation inputter 11 is provided with various switches, a button, a track pad, a track ball, a mouse, a keyboard, a touch panel that is integrally provided on a display screen of the display 16 and detects a touch operation on the display screen and the like. The operation inputter 11 inputs a command for indicating diagnosis start, data such as personal information of the subject, and various parameters for displaying the ultrasonic image on the display 16, for example. Then, the operation inputter 11 outputs an operation signal corresponding to the input operation to the controller 19.

The transmitter 12 is a circuit that supplies the drive signal that is the electric signal to the ultrasonic probe 2 via the cable 3 to cause the ultrasonic probe 2 to generate the transmission ultrasonic wave under control of the controller 19.

The transmitter 12 is provided with, for example, a clock generation circuit, a delay circuit, and a pulse generation circuit. The clock generation circuit is a circuit that generates a clock signal that determines a transmission timing and a transmission frequency of the drive signal. The delay circuit is a circuit for setting a delay time for each individual path corresponding to each vibrator 2a, delaying the transmission of the drive signal by the set delay time, and performing focusing of a transmission beam formed of the transmission ultrasonic wave (transmission beam forming) and the like. The pulse generation circuit is a circuit for generating a pulse signal as the drive signal at set voltage and time interval.

Under the control of the controller 19, the transmitter 12 configured as described above sequentially switches a plurality of vibrators 2a to which the drive signal is supplied while shifting them by a predetermined number each time the ultrasonic wave is transmitted and received, and performs scanning (scanning) by supplying the drive signal to a plurality of vibrators 2a the output of which is selected.

The receiver 13 is a circuit that receives the reception signal that is the electric signal from the ultrasonic probe 2 via the cable 3 under the control of the controller 19. The receiver 13 is provided with, for example, an amplifier, an A/D conversion circuit, and a phasing addition circuit. Note that the transmitter 12 and the receiver 13 serve as a “transmitter/receiver” of the present invention.

The amplifier is a circuit for amplifying the reception signal with an amplification factor set in advance for each individual path corresponding to each vibrator 2a. The A/D conversion circuit is a circuit for performing analog-digital conversion (A/D conversion) on the amplified reception signal. The phasing addition circuit is a circuit for giving a delay time to the A/D-converted reception signal for each individual path corresponding to each vibrator 2a to adjust a time phase, and adds them (phasing addition) to generate sound ray data. That is, the phasing addition circuit performs reception beam forming on the reception signal for each vibrator 2a to generate the sound ray data (corresponding to “reception data” of the present invention), and outputs the generated sound ray data to the image generator 14 under the control of the controller 19.

Under the control of the controller 19, the image generator 14 performs envelope detection processing, logarithmic compression and the like on the sound ray data output from the receiver 13, and performs luminance conversion by adjusting a dynamic range and a gain, thereby generating brightness (B) mode image data (hereinafter, ultrasonic image data) as tomographic image data. That is, the ultrasonic image data indicates intensity of the reception signal by luminance. The controller 19 acquires the ultrasonic image data generated by the image generator 14 and causes the storage 17 to store the same.

The image generator 14 inputs the imaged data output from the imager 4 and generates imaged image data. The controller 19 acquires the imaged image data generated by the image generator 14 and causes the storage 17 to store the same. Note that the image generator 14 serves as an “ultrasonic image generator” and an “imaged image generator” of the present invention.

The voice outputter 15 formed of, for example, a speaker, notifies a user (operator) of information by voice from the ultrasonic image diagnostic device 100.

The display 16 is a display device such as a liquid crystal display (LCD), a cathode-ray tube (CRT) display, an organic electronic luminescence (EL) display, an inorganic EL display, and a plasma display. The display 16 displays the ultrasonic image corresponding to the ultrasonic image data generated by the image generator 14 on a display screen under the control of the controller 19. The display 16 displays an imaged image corresponding to the imaged image data generated by the image generator 14 on the display screen under the control of the controller 19.

The storage 17 is a storage capable of writing and reading information such as a flash memory, a hard disk drive (HDD), and a solid state drive (SSD). The storage 17 stores the ultrasonic image data generated by the image generator 14 in time series. The storage 17 also stores the imaged image data generated by the image generator 14 in time series. The storage 17 also stores the voice data output from the voice inputter 5 to the controller 19 in time series. That is, the storage 17 serves as an “ultrasonic image storage”, an “imaged image storage”, and a “voice data storage” of the present invention.

The moving image generator 18 acquires the ultrasonic image data, the imaged image data, and the voice data from the storage 17 in time series, and generates moving image data including the voice. Then, the moving image generator 18 outputs the generated moving image data to the controller 19. Note that the moving image generator 18 may acquire the ultrasonic image data and the imaged image data from the storage 17 in time series and generate moving the image data not including the voice. The moving image generator 18 may acquire the ultrasonic image data from the storage 17 in time series and generate the moving image data not including the voice.

The controller 19 is provided with, for example, a central processing unit (CPU), a read only memory (ROM), and a random access memory (RAM), reads various processing programs such as a system program stored in the ROM, expands the same on the RAM, and performs centralized control of the operation of each unit of the ultrasonic image diagnostic device main body 1 according to the expanded programs.

The ROM is formed of a nonvolatile memory and the like such as a semiconductor, and stores a system program corresponding to the ultrasonic image diagnostic device 100, various processing programs executable on the system program, various data such as a gamma table and the like. These programs are stored in a form of computer readable program codes, and the CPU sequentially executes operations according to the program codes. The RAM forms a work area in which various programs executed by the CPU and data related to these programs are temporarily stored. In this embodiment, a “display control program” is stored in the ROM of the controller 19.

In a case where diagnosis of the ultrasonic image is performed by the user, the controller 19 controls the display 16 to simultaneously display the ultrasonic image and the imaged image corresponding to the ultrasonic image data and the imaged image data generated by the image generator 14, respectively, in real time. In this embodiment, the controller 19 displays a combined image obtained by combining the ultrasonic image and the imaged image in real time.

In a case where the diagnosis of the ultrasonic image is performed by the user, the controller 19 sequentially acquires the ultrasonic image data generated by the image generator 14 and causes the storage 17 to store the same. In a case where the diagnosis of the ultrasonic image is performed by the user, the controller 19 sequentially acquires the imaged image data generated by the image generator 14 and causes the storage 17 to store the same. In a case where the diagnosis of the ultrasonic image is performed by the user, the controller 19 sequentially acquires the voice data output from the voice inputter 5 and causes the storage 17 to store the same.

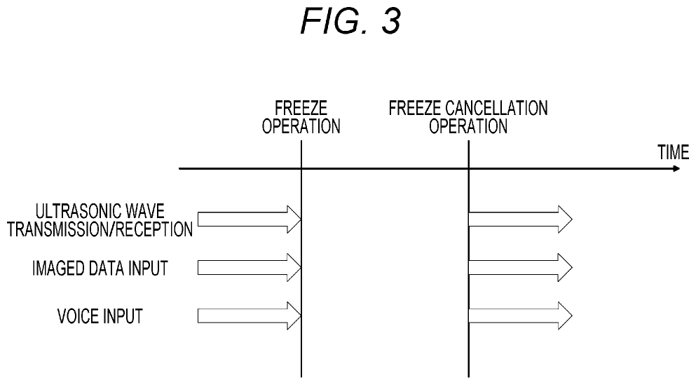

In a case where a freeze instruction of the ultrasonic image is generated on the basis of an operation signal (freeze operation) output from the operation inputter 11 during real-time display of the ultrasonic image and the imaged image, the controller 19 stops the reception of the ultrasonic wave by the receiver 13, the input of the imaged data by the image generator 14, and the reception of the input of the voice by the voice inputter 5 (refer to FIG. 3). As a result, the real-time display of the ultrasonic image and the imaged image is stopped, and the storage of the ultrasonic image data, the imaged image data, and the voice data in the storage 17 is stopped.

In a case where a freeze cancellation instruction of the ultrasonic image is generated on the basis of an operation signal (freeze cancellation operation) output from the operation inputter 11 after the freeze instruction of the ultrasonic image is generated, the controller 19 restarts the reception of the ultrasonic wave by the receiver 13, the input of the imaged data by the image generator 14, and the reception of the input of the voice by the voice inputter 5 (refer to FIG. 3). As a result, the real-time display of the ultrasonic image and the imaged image is restarted, and the storage of the ultrasonic image data, the imaged image data, and the voice data in the storage 17 is restarted.

In a case where the diagnosis of the ultrasonic image is not performed by the user, when a moving image reproduction instruction is generated on the basis of an operation signal output from the operation inputter 11, the controller 19 outputs the moving image data generated by the moving image generator 18 to the voice outputter 15 and the display 16 to reproduce a moving image corresponding to the moving image data.

In a case where the freeze instruction of the ultrasonic image is generated on the basis of the operation signal (freeze operation) output from the operation inputter 11 during the reproduction of the moving image, the controller 19 stops the output of the moving image data to the voice outputter 15 and the display 16, and eventually the reproduction of the moving image.

In a case where the freeze cancellation instruction of the ultrasonic image is generated on the basis of the operation signal (freeze cancellation operation) output from the operation inputter 11 while the reproduction of the moving image is stopped, the controller 19 restarts the output of the moving image data to the voice outputter 15 and the display 16, and eventually the reproduction of the moving image.

In a case where the diagnosis of the ultrasonic image is not performed by the user, when a display instruction of the ultrasonic image is generated on the basis of an operation signal (cine operation) output from the operation inputter 11, the controller 19 acquires an ultrasonic image (cine image) and an imaged image generated at the same timing from the storage 17 and simultaneously displays them, and acquires the voice data acquired at the same timing from the storage 17 and outputs the same.

As described above in detail, in this embodiment, the ultrasonic image diagnostic device 100 is provided with the transmitter/receiver (transmitter 12 and receiver 13) that drives the ultrasonic probe 2 to transmit the ultrasonic wave into the subject and receives the ultrasonic wave from the inside of the subject to output the reception data, the ultrasonic image generator (image generator 14) that generates the ultrasonic image on the basis of the output reception data, the imaged image generator (image generator 14) that inputs the imaged data output from the imager 4 that images the periphery of the subject and generates the imaged image, and the controller 19 that simultaneously displays the generated ultrasonic image and imaged image in real time.

According to this embodiment configured in this manner, the generated ultrasonic image and imaged image are simultaneously displayed in real time (with matching time series). Therefore, for example, in a case where the imaged image is utilized as a body mark, the user may easily grasp in real time an operation status of the ultrasonic probe 2 (probe) and a status of the subject (appearance information of the subject and a site to be diagnosed), that is, information inside and outside the subject in addition to the ultrasonic image, and may eventually improve workability and accuracy of ultrasonic diagnosis. In contrast, in the conventional technology (the technology disclosed in JP 2001-112752 A), in a case where it is desired to grasp the information inside and outside the subject in real time, it is required to directly visually confirm the operation status of the ultrasonic probe 2 (probe) and the status of the subject, and there is a problem that it takes time and effort.

In this embodiment, the imager 4 is connected to the ultrasonic image diagnostic device main body 1, the input of the imaged data and the reception of the input of the voice are performed live in a live state of the ultrasonic image, the input of the imaged data and the reception of the input of the voice are frozen when the ultrasonic image is frozen, and freezing of the input of the imaged data and the reception of the input of the voice is canceled (transition to the live state) when the freezing of the ultrasonic image is canceled (transition to the live state). As a result, the ultrasonic image, the imaged image, and the voice in the live state in the ultrasonic diagnosis may be recorded as a still image or a moving image with matching time series. Therefore, the ultrasonic image, the imaged image, and the voice acquired by the ultrasonic image diagnostic device 100 may be suitably utilized as a record of an ultrasonic diagnosis (inspection) situation.

Note that, in the above-described embodiment, in a case where the freeze instruction of the ultrasonic image is generated on the basis of the operation signal (freeze operation) output from the operation inputter 11 during real-time display of the ultrasonic image and the imaged image, the controller 19 does not have to stop the reception of the input of the voice by the voice inputter 5 while this stops the reception of the ultrasonic wave by the receiver 13 and the input of the imaged data by the image generator 14. In this case, in a case where the freeze cancellation instruction of the ultrasonic image is generated on the basis of the operation signal (freeze cancellation operation) output from the operation inputter 11 after the freeze instruction of the ultrasonic image is generated, the controller 19 restarts the reception of the ultrasonic wave by the receiver 13 and the input of the imaged data by the image generator 14.

In the above-described embodiment, in a case where the diagnosis of the ultrasonic image is not performed by the user, when the display instruction of the ultrasonic image is generated on the basis of the operation signal (cine operation) output from the operation inputter 11, the controller 19 acquires the ultrasonic image and the imaged image generated at the same timing from the storage 17 and simultaneously displays them, but this does not have to acquire the voice data acquired at the same timing from the storage 17 and output the same.

In the above-described embodiment, in a case where the diagnosis of the ultrasonic image is performed by the user, the controller 19 may control the display 16 to display in real time only the ultrasonic image corresponding to the ultrasonic image data generated by the image generator 14, for example, temporarily. As a result, the user may intensively confirm the ultrasonic image displayed on the display 16. In a case where the diagnosis of the ultrasonic image is performed by the user, the controller 19 may control the voice inputter 5 and the display 16, and stop the output of the voice data and display in real time only the ultrasonic image corresponding to the ultrasonic image data generated by the image generator 14, for example, temporarily.

In a case where the diagnosis of the ultrasonic image is not performed by the user, when the moving image reproduction instruction is generated on the basis of the operation signal output from the operation inputter 11, the controller 19 may output the moving image data generated by the moving image generator 18 to the voice outputter 15 to reproduce the moving image corresponding to the moving image data (silent moving image not including the voice), for example, temporarily.

In the above-described embodiment, in a case where the diagnosis of the ultrasonic image is not performed by the user, when the display instruction of the ultrasonic image is generated on the basis of the operation signal (cine operation) output from the operation inputter 11, the controller 19 acquires the ultrasonic image and the imaged image generated at the same timing from the storage 17 and simultaneously displays them, but this does not have to acquire the voice data acquired at the same timing from the storage 17 and output the same.

In the above-described embodiment, the image generator 14 may be provided with an image memory (not illustrated) formed of a semiconductor memory such as a dynamic random access memory (DRAM), and may store the generated ultrasonic image data in the image memory in units of frames. In this case, the image generator 14 performs image processing such as image filter processing or time smoothing processing on the ultrasonic image data read from the image memory, and performs scan conversion to a display image pattern to be displayed on the display 16.

In the above-described embodiment, a part or all of the functions of the respective functional blocks of the transmitter 12, the receiver 13, the image generator 14, the moving image generator 18, and the controller 19 included in the ultrasonic image diagnostic device 100 may be realized as a hardware circuit such as an integrated circuit. The integrated circuit is a large scale integration (LSI), for example; the LSI is sometimes referred to as an IC, a system LSI, a super LSI, and an ultra LSI depending on an integration degree. A method of realizing the integrated circuit is not limited to the LSI; this may also be realized by a dedicated circuit or a general-purpose processor, and a field programmable gate array (FPGA) and a reconfigurable processor capable of reconfiguring connection and setting of a circuit cell in the LSI may also be used. A part or all of the functions of each functional block may also be executed by software. In this case, the software is stored in one or more of storage media such as a ROM, an optical disk, or a hard disk, and the software is executed by an arithmetic processor.

The above-described embodiment merely describes an example of substantiation when carrying out the present invention, and the technical scope of the present invention cannot be interpreted in a limited manner by the same. That is, the present invention may be variously carried out without departing from the gist or the main characteristics thereof.

Although embodiments of the present invention have been described and illustrated in detail, the disclosed embodiments are made for purposes of illustration and example only and not limitation. The scope of the present invention should be interpreted by terms of the appended claims

What is claimed is:

1. An ultrasonic image diagnostic device comprising:
a transmitter/receiver that drives an ultrasonic probe to transmit an ultrasonic wave into a subject and receives the ultrasonic wave from an inside of the subject to output reception data;
an ultrasonic image generator that generates an ultrasonic image on the basis of the output reception data;
an imaged image generator that inputs imaged data output from an imager that images a periphery of the subject, and generates an imaged image; and
a hardware processor that simultaneously displays the generated ultrasonic image and imaged image in real time.

2. The ultrasonic image diagnostic device according to claim 1, wherein
the hardware processor stops the reception of the ultrasonic wave and the input of the imaged data in a case where a freeze instruction of the ultrasonic image is generated.

3. The ultrasonic image diagnostic device according to claim 2, wherein
the hardware processor restarts the reception of the ultrasonic wave and the input of the imaged data in a case where a freeze cancellation instruction of the ultrasonic image is generated.

4. The ultrasonic image diagnostic device according to claim 1, comprising:
a voice data acquirer that acquires voice data output from a voice inputter that receives an input of voice, wherein
the hardware processor stops the reception of the ultrasonic wave, the input of the imaged data, and the reception of the input of the voice in a case where a freeze instruction of the ultrasonic image is generated.

5. The ultrasonic image diagnostic device according to claim 4, wherein
the hardware processor restarts the reception of the ultrasonic wave, the input of the imaged data, and the reception of the input of the voice in a case where a freeze cancellation instruction of the ultrasonic image is generated.

6. The ultrasonic image diagnostic device according to claim 1, comprising:
a moving image generator that generates a moving image by acquiring the generated ultrasonic image and imaged image in time series.

7. The ultrasonic image diagnostic device according to claim 4, comprising:
a moving image generator that acquires the generated ultrasonic image and imaged image, and the acquired voice data in time series, and generates a moving image including the voice.

8. The ultrasonic image diagnostic device according to claim 6, wherein
the hardware processor reproduces the generated moving image.

9. The ultrasonic image diagnostic device according to claim 8, wherein
the hardware processor stops the reproduction of the moving image in a case where a freeze instruction of the ultrasonic image is generated.

10. The ultrasonic image diagnostic device according to claim 9, wherein
the hardware processor restarts the reproduction of the moving image in a case where a freeze cancellation instruction of the ultrasonic image is generated.

11. The ultrasonic image diagnostic device according to claim 1, comprising:
an ultrasonic image storage that stores the generated ultrasonic image in time series; and
an imaged image storage that stores the generated imaged image in time series, wherein
the hardware processor acquires the ultrasonic image and the imaged image generated at the same timing from the ultrasonic image storage and the imaged image storage, respectively, and simultaneously displays the ultrasonic image and the imaged image.

12. The ultrasonic image diagnostic device according to claim 4, comprising:
an ultrasonic image storage that stores the generated ultrasonic image in time series;
an imaged image storage that stores the generated imaged image in time series; and
a voice data storage that stores the acquired voice data in time series, wherein
the hardware processor acquires the ultrasonic image and the imaged image generated at the same timing from the ultrasonic image storage and the imaged image storage, respectively, and simultaneously displays the ultrasonic image and the imaged image, and acquires the voice data acquired at the same timing from the voice data storage and outputs the voice data.

13. The ultrasonic image diagnostic device according to claim 1, wherein
the hardware processor displays only the generated ultrasonic image in real time.

14. The ultrasonic image diagnostic device according to claim 4, wherein
the hardware processor stops outputting the voice data and displays only the ultrasonic image in real time.

15. A display control method comprising:
driving an ultrasonic probe to transmit an ultrasonic wave into a subject and receiving the ultrasonic wave from an inside of the subject to output reception data;
generating an ultrasonic image on the basis of the output reception data;
inputting imaged data output from an imager that images a periphery of the subject, and generating an imaged image; and
simultaneously displaying the generated ultrasonic image and imaged image in real time.

16. A non-transitory recording medium storing a computer readable display control program that causes a computer to execute:
driving an ultrasonic probe to transmit an ultrasonic wave into a subject and receiving the ultrasonic wave from an inside of the subject to output reception data;
generating an ultrasonic image on the basis of the output reception data;
inputting imaged data output from an imager that images a periphery of the subject, and generating an imaged image; and
simultaneously displaying the generated ultrasonic image and imaged image in real time.
